(19) United States
(12) Patent Application Publication
(10) Pub. No.: US 2001/0007874 A1
(43) Pub. Date: Jul. 12, 2001

(54) Pesticidal compositions

(76) Inventors: Michael Oostendorp, Rheinfelden (DE); Wilhelm Ruess, Pfeffingen (CH)

Correspondence Address: SYNGENTA, 3054 CORNWALLIS ROAD, RESEARCH TRIANGLE PARK, NC, 27709, US

(21) Appl. No.: 09/771,090

(22) Filed: Jan. 27, 2001

Related U.S. Application Data
(63) Continuation of application No. PCT/EP99/05415, filed on Jul. 28, 1999.

(30) Foreign Application Priority Data
Jul. 30, 1998 (GB) 9816641.6

(51) Int. Cl. A61K 31/41; A01N 43/82

(52) U.S. Cl. 514/361

(57) ABSTRACT

A composition and method for protecting and immunizing plants against attack by microorganisms, comprising at least two active ingredient components in amounts having synergistic action, together with a suitable carrier, wherein component I is a compound of formula

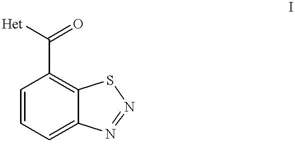

in which

Het is an optionally substituted 5- or 6-membered aromatic ring having 1-3 hetero atoms N, O and/or S; in particular selected from the group

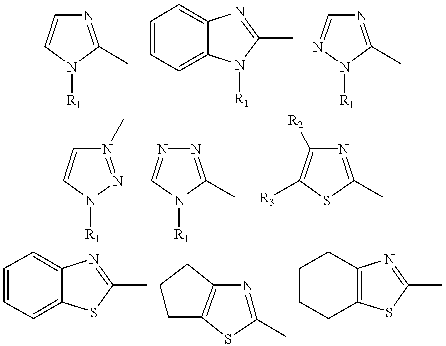

wherein

R₁ is hydrogen, optionally substituted C₁-C₆alkyl, optionally substituted C₂-C₆alkenyl or optionally substituted phenyl and

R₂ and R₃ independently of each other are hydrogen or optionally substitued C₁-C₆alkyl, and acid addition salts and metal salt complexes thereof;

and wherein component II is the fungicide carpropamide.

This is a continuation of International Application No. PCT/EP99/05415, filed Jul. 28, 1999, the contents of which are incorporated herein by reference.

The present invention relates to new compositions and methods for protecting and immunizing plants against attack by microorganisms, such as phytopathogenic fungi, bacteria and viruses and for improving the qualities of the plants.

The compositions comprise at least two active ingredient components in an effective amount together with a suitable carrier, wherein component I is a compound of formula I

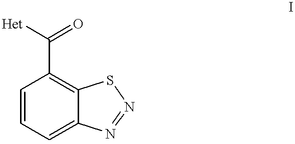

in which

Het is an optionally substituted 5- or 6-membered aromatic ring having 1-3 hetero atoms N, O and/or S; in particular selected from the group

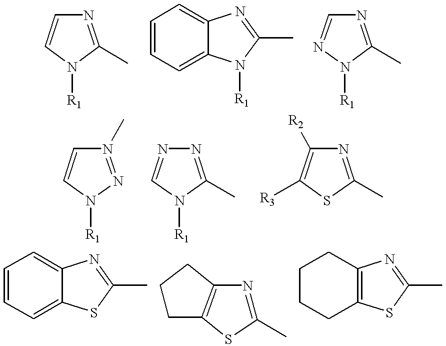

wherein

R₁ is hydrogen, optionally substituted C₁-C₆alkyl, optionally substituted C₂-C₆alkenyl or optionally substituted phenyl and

R₂ and R₃ independently of each other are hydrogen or optionally substitued C₁-C₆alkyl, and acid addition salts and metal salt complexes thereof (EP-A-816,363);

and wherein component II is the fungicide carpropamide (KTU3616; trans-2,2-dichloro-N-[1-(4-chlorophenyl)ethyl]-1-ethyl-3-methyl-cyclopropanecarboxamide) (The Pesticide Manual, 11th. Ed. 1997, No. 440).

It is known from EP-A-816,363 that compounds of formula I are active as microbicides and may be used in agriculture and horticulture for directly combating undesired phytopathogenic fungi and bacteria and for immunizing plants against attack of phytopathogenic fungi and bacteria by inducing “Systemic Activated Resistance” (SAR) in the plants. Surprisingly it has now been found that compositions and methods according to the invention exhibit synergistic activities and are therefore particularly effective for protecting and immunizing plants against attack by microorganisms, such as phytopathogenic fungi, bacteria and viruses.

In the above formula alkyl designates straight chain and branched alkyl groups, such as methyl, ethyl, n-propyl, i-propyl, n-butyl, s-butyl, i-butyl, t-butyl, n-pentyl, i-pentyl, s-pentyl, neo-pentyl, and the various isomers of hexyl.

Alkenyl is straight-chain or branched alkenyl such as allyl, methallyl, 1 -methylvinyl or but-2-en-1-yl. Preferred alkenyl radicals contain 3 to 4 carbon atoms in the chain. Substituents at alkyl, alkenyl and phenyl include halogen, such as fluoro, chloro, bromo and iodo; cyano; alkoxy, such as methoxy, ethoxy, n-propoxy, i-propoxy, and the various isomers of butoxy; alkylthio, such as methylthio and ethylthio; alkoxycarbonyl, such as methoxycarbonyl and ethoxycarbonyl; and phenyl.

Preferred compounds of formula I are those wherein

R₁ is hydrogen, C₁-C₄alkyl, C₁-C₄haloalkyl, phenyl-C₁-C₄alkyl, cyano- C₁-C₄alkyl, C₁-C₂alkoxy-C₁₋₄alkyl, C₁-C₂alkythio- C₁-C₄alkyl, C₁-C₂alkoxycarbonyl- C₁-C₄alkyl, C₂-C₄alkenyl, phenyl, halogen substituted phenyl or C₁-C₄alkyl substituted phenyl and

R₂ and R₃ independently of each other are hydrogen or C₁-C₄alkyl.

Particularly preferred are those, wherein

R₁ is hydrogen, methyl, ethyl, propyl, isopropyl, butyl, cyanomethyl, 2-cyanoethyl, 1-cyanoethyl, 3-cyano-propyl, methoxymethyl, ethoxymethyl, methylthiomethyl, methoxycarbonyl-ethyl, ethoxycarbonyl-methyl, ethoxycarbonyl-ethyl, fluoromethyl, difluoromethyl, trifluoromethyl, chloromethyl, benzyl, phenyl, chlorophenyl, methylphenyl, vinyl, allyl or but-3-en-1-yl, and

R₂ and R₃ independently of each other are hydrogen, methyl, ethyl, propyl or isopropyl. Especially preferred are the specific compounds as disclosed in EP-A-816,363. Suitable acid addition salts and metal salts complexes thereof are also described in this reference.

The compositions of the invention can be used in the agricultural sector and related fields preventively and/or curatively. Plants can be protected both by direct action on the pests or by activation and stimulation of the plant's own defense system (immunization). The latter mode of action has also become known by the name “Systemic Activated Disease Resistance” (“SAR”).

Accordingly, using the mixtures of the invention, it is possible to control plant diseases on the one hand by strengthening the plant by activating its own defence system and on the other hand by additionally controlling the pathogens directly.

A particular advantage of the mixtures according to the invention is further that, because the modes of action of components I and II are completely different, the threat of resistance being developed in the control of plant diseases is effectively prevented.

The compositions can also be used as dressings in the treatment of seed (fruit, tubers, grains) and plant cuttings to provide protection.

Advantageous mixing ratios of the two active ingredients are I:II=300:1 to 1:100, 100:1 to 1:50, 10:1 to 1:10, and preferably 1:5 to 1:3.

The compositions are effective against the following phytopathogenic pests:

A) Fungi, for example: Ascomycetes (e.g. Venturia, Podosphaera, Erysiphe, Monilinia, Mycosphaerella, Uncinula); Basidiomycetes (e.g. the genus Hemileia, Rhizoctonia, Puccinia); Fungi imperfecti (e.g. Botrytis, Helminthosporium, Rhynchosporium, Fusarium, Septoria, Cercospora, Alternaria, Pyricularia and Pseudocercosporella herpotrichoides); Oomycetes (e.g. Phytophthora, Peronospora, Bremia, Pythium, Plasmopara);

B) Bacteria, for example Pseudomonoadaceae (e.g. Xanthomonas, Pseudomonas), Rhizobiaceae, Enterobacteriaceae, Corynebacteriaceae, Streptomycetaceae;

C) Viruses, for example, Rod-shaped Viruses (e.g. TMV, TRV, Carlavirus, BYV, PVX, PVY), Sphaeric Viruses (e.g. Tymovirus, Nepovirus, TRV, TNV, CMV, Comovirus, Tombusvirus), Alfalfa Mosaic Virus, Diplornaviruses (e.g. Tomato spotted wilt virus, Rhabdovirus), Cauliflower Mosaic Virus, Rhizomania.

Quality of plants means, for example, shape and size, flavor and taste, colour, imperishable nature, content of useful ingredients, as nutritive substance, vitamins etc.

Target crops to be activated within the scope of the present invention comprise e.g. the following species of plants: cereals (wheat, barley, rye, oats, rice, maize, sorghum and related species); beet (sugar beet and fodder beet); pomes, stone fruit and soft fruit (apples, pears, plums, peaches, almonds, cherries, mirabella, mangos strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (marrows, cucumber, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); and plants such as tobacco, nuts, coffee, aubergines, sugar cane, tea, pepper, vines, hops, bananas and natural rubber plants, as well as ornamentals (flowers, shrubs, broad-leaved trees and evergreens, such as conifers). This list does not represent any limitation.

The compositions are particularly useful for controlling/treating the following pests/crops:

Pyricularia oryzeae in rice, in particular for seedling box-, into water granules- and for foliar application;

Xanthomonas and Pseudomonas in tomatoes, in particular for foliar application;

Potatoe Y-virus in potatoes, in particular for foliar application;

Rhizomania on sugar beet, in particular for seed treatment;

Colletotrichum lagenarium on vegetables, in particular for foliar application.

The method of the invention comprises applying to the plants or to the locus thereof in admixture or separately, an effective amount of a component I and a component II in any desired sequence or simultaneously.

The term locus as used herein is intended to embrace the fields on which the treated crop plants are growing, or where the seeds of cultivated plants are sown, or the place where the seed will be placed into the soil. The term seed is intended to embrace plant propagating material such as cuttings, seedlings, seeds, germinated or soaked seeds.

A method of applying the composition is application to the leaves (foliar application). The frequency and rate of application depend upon the risk of infestation by the corresponding pathogen. The compounds can also penetrate the plant through the roots via the soil (systemic action) if the locus of the plant is impregnated with a liquid formulation or if the substances are introduced in solid form into the soil, e.g. in the form of granules (soil application). In paddy rice crops, such granules can be applied in metered amounts to the flooded rice field or to the seedling box before transplanting. In order to treat seed, the compounds I can, however, also be applied to the seeds (coating), either by impregnating the grains or tubers with a liquid formulation of the active ingredient, or by coating them with a solid formulation.

The compounds are used together with the adjuvants conventionally employed in formulation technology. For that purpose they are advantageously formulated in known manner e.g. into emulsifiable concentrates, coatable pastes, directly sprayable or dilutable solutions, dilute emulsions, wettable powders, soluble powders, dusts, granules or by encapsulation in e.g. polymer substances. As with the nature of the compositions, the methods of application, such as spraying, atomizing, dusting, scattering, coating or pouring, are chosen in accordance with the intended objectives and the prevailing circumstances.

Advantageous rates of application of the active ingredient mixture are normally from 0.01 to 10 kg of active ingredient (a.i.) per hectare, preferably from 30 g to 1000 g a.i./ha, especially from 50 g to 500 g a.i./ha. In the case of the treatment of seed, the rates of application are from 0.5 g to 1000 g, preferably from 5 g to 100 g, a.i. per 100 kg of seed.

The agrochemical compositions generally comprise 0.1 to 99% by weight, preferably 0.1 to 95% by weight, of a compound of formula 1, 99.9 to 1% by weight, preferably 99.8 to 5% by weight, of a solid or liquid adjuvant and 0 to 25% by weight, preferably 0.1 to 25% by weight, of a surfactant. The compositions may also comprise further auxiliaries, such as stabilizers, antifoams, viscosity regulators, binders or tackifiers, as well as fertilizers or other active ingredients for obtaining special effects. Manufacturing of the formulations is a routine matter in the art of pesticides and largely depends on the desired mode of application and target plant.

### EXAMPLES

### Formulation Examples

| Wettable powders | a) | b) | c) |
|---|---|---|---|
| active ingredient (I:II = 1:3) | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| kaolin | 62% | 27% | — |

The active ingredient is thoroughly mixed with the adjuvants and the mixture is thoroughly ground in a suitable mill, affording wettable powders which can be diluted with water to give suspensions of the desired concentration.

Emulsifiable concentrate

| | |
|---|---|
| active ingredient (I:II) = 1:6) | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| cyclohexanone | 30% |
| xylene mixture | 50% |

Emulsions of any required dilution, which can be used in plant protection, can be obtained from this concentrate by dilution with water.

Extruder granules

| | |
|---|---|
| active ingredient (I:II = 2:1) | 15% |
| sodium lignosulfonate | 2% |
| carboxymethylcellulose | 1% |
| kaolin | 82% |

The active ingredient is mixed and ground with the adjuvants, and the mixture is moistened with water. The mixture is extruded and then dried in a stream of air.

Coated granules

| | |
|---|---|
| active ingredient (I II = 1:2) | 8% |
| polyethylene glycol (mol. wt. 200) | 3% |
| kaolin | 89% |

The finely ground active ingredient is uniformly applied, in a mixer, to the kaolin moistened with polyethylene glycol. Non-dusty coated granules are obtained in this manner.

### Slow Release Capsule Suspension

28 parts of a combination of the compounds I and II or of each of these compounds separately, are mixed with 2 parts of an aromatic solvent and 7 parts of toluene diisocyanate/polymethylene-polyphenylisocyanate-mixture (8:1). This mixture is emulsified in a mixture of

1.2 parts of polyvinylalcohol, 0.05 parts of a defoamer and 51.6 parts of water until the desired particle size is achieved. To this emulsion a mixture of 2.8 parts

1,6-diaminohexane in 5.3 parts of water is added. The mixture is agitated until the polymerisation reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

### Seed Dressing Formulation

25 parts of a combination of the compounds I and II, 15 parts of dialkylphenoxypoly(ethylenoxy)ethanol, 15 parts of fine silica, 44 parts of fine kaolin, 0.5 parts of Rhodamine B as a colorant and 0.5 parts of Xantham Gum are mixed and ground in a contraplex mill at approx. 10000 rpm to an average particle size of below 20 microns. The resulting formulation is applied to seeds as an aqueous suspension in an apparatus suitable for that purpose.

### Biological Examples

A synergistic effect exists whenever the action of an active ingredient combination is greater than the sum of the actions of the individual components.

The action to be expected E for a given active ingredient combination obeys the so-called COLBY formula and can be calculated as follows:

ppm=milligrams of active ingredient (=a.i.) per liter of spray mixture

X=% action by active ingredient I using p ppm of active ingredient

Y=% action by active ingredient II using q ppm of active ingredient.

According to Colby, the expected (additive) action of active ingredients I+II using p+q ppm of active ingredient is

$$E = X + Y - \frac{X \cdot Y}{100}$$

If the action actually observed (O) is greater than the expected action (E), then the action of the combination is superadditive, i.e. there is a synergistic effect.

O/E=synergy factor (SF).

### Example B-1

### Action Against Pyricularia oryzae on Rice Plants

3 week old rice plants cv. Sasanishiki were treated with the formulated test compounds or a mixture thereof in a spray chamber. Two days after application rice plants were inoculated by spraying a spore suspension (3×10⁵ conidia/ml) on the test plants. After an incubation period of 6 days at 25° C. and 95% relative humidity the disease incidence was assessed.

### Example B-2

### Immunization of Cucumis sativus L. Against Colletotrichum lagenarium

a) After a cultivation period of 2 weeks, cucumber plants are sprayed with a spray mixture prepared from a wettable powder formulation of the test compounds or a mixture thereof. After 72 hours, the plants are infected with a spore suspension (1.0×10⁵ spores/ml) of the fungus and incubated for 30 hours at high humidity and a temperature of 23° C. Incubation is then continued at normal humidity and 22° C. to 23° C.

Evaluation of protective action is made 7 to 8 days after infection and is based on fungus infestation.

b) After a cultivation period of 2 weeks, cucumber plants are treated by soil application with a spray mixture prepared from a wettable powder formulation of the test compounds or a mixture thereof. After 72 hours, the plants are infected with a spore suspension (1.5× 10⁵ spores/ml) of the fungus and incubated for 30 hours at high humidity and a temperature of 23° C. Incubation is then continued at normal humidity and 22° C.

Evaluation of protective action is made 7 to 8 days after infection and is based on fungus infestation.

The composition exhibit good activity in tests (a) and (b) and reduce fungus infestation to 0 to 20%. On the other hand, Colletotrichum infestation is 90% on untreated and infected control plants.

1. A composition for protecting and immunizing plants against attack by microorganisms, comprising at least two active ingredient components in amounts having synergistic action, together with a suitable carrier, wherein component I is a compound of formula 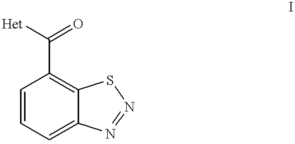
in which
Het is an optionally substituted 5- or 6-membered aromatic ring having 1-3 hetero atoms N, O and/or S; in particular selected from the group 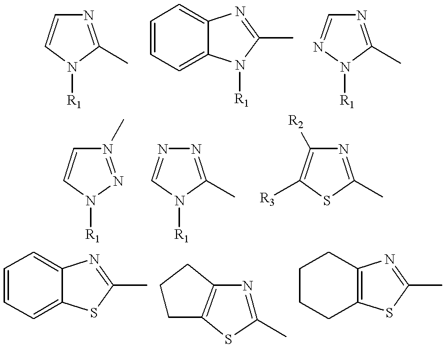
wherein
R₁ is hydrogen, optionally substituted C₁-C₆alkyl, optionally substituted C₂-C₆alkenyl or optionally substituted phenyl and
R₂ and R₃ independently of each other are hydrogen or optionally substitued C₁-C₆alkyl, and acid addition salts and metal salt complexes thereof;
and wherein component II is the fungicide carpropamide.

2. A method of protecting plants against microorganisms by treating the plants, parts of the plants or their locus with an effective amount of component I and component II according to claim 1, in any desired sequence or simultaneously.

3. A method according to claim 2 wherein rice, potatoes, tomatoes and sugar beet are treated.

4. A method according to claim 2 wherein seed is treated.
